(19) United States
(12) Patent Application Publication
(10) Pub. No.: US 2008/0064981 A1
(43) Pub. Date: Mar. 13, 2008

(54) METHOD AND APPARATUS FOR DETERMINING ELECTRICAL PROPERTIES OF OBJECTS CONTAINING INHOMOGENEITIES

(76) Inventor: Christopher Gregory, Milwaukee, WI (US)

Correspondence Address: QUARLES & BRADY LLP, 411 E. WISCONSIN AVENUE, SUITE 2040, MILWAUKEE, WI, 53202-4497, US

(21) Appl. No.: 11/857,493

(22) Filed: Sep. 19, 2007

Related U.S. Application Data
(63) Continuation-in-part of application No. 10/700,876, filed on Nov. 4, 2003.
(60) Provisional application No. 60/845,839, filed on Sep. 19, 2006.
(60) Provisional application No. 60/424,568, filed on Nov. 7, 2002.

(51) Int. Cl. A61B 5/053

(52) U.S. Cl. 600/547

(57) ABSTRACT

An electrical parameter imaging apparatus and method includes the acquisition of a charge distribution pattern on an array of electrodes that surround an object being imaged. In addition the boundaries of regions having differing electrical characteristics within the object are measured by a secondary imaging method. The internal boundary location measurements are employed to provide a quicker method to compute electrical parameters of tissues inside the boundary using the acquired charge distribution pattern.

### CROSS-REFERENCE TO RELATED APPLICATIONS

This application claims the benefit of U.S. Provisional Application No. 60/845,839, filed on Sep. 19, 2006, and is a continuation-in-part of U.S. patent application Ser. No. 10/700,876 filed on Nov. 4, 2003, and titled “Method And Apparatus For Producing An Electrical Property Image Of Substantially Homogeneous Objects Containing Inhomogeneities” which claims the benefit of U.S. Provisional Application No. 60/424,568, filed on Nov. 7, 2002.

### BACKGROUND OF THE INVENTION

This invention relates to electrical imaging technology, and more specifically to an apparatus and method for computing accurate values of the electrical properties of objects with substantially reduced processing time. More specifically, a structural image of the object is acquired to locate boundaries therein between different tissues, and then a series of measurements are made in which voltages are applied to the surface of the object and resulting surface currents are measured. An image is reconstructed using this information which includes: predicted currents at an internal boundary; and calculating a first contrast ratio at the internal boundary which indicates the relationship between the electrical characteristics of the adjacent tissues. From the contrast ratio and the known electrical characteristics of the tissue on one side of the boundary, the electrical characteristics of the tissue on the other side of the boundary are calculated. These calculations are made at successive boundaries using previously calculated electrical characteristics.

The demand for new medical imaging modalities is driven by the need to identify tissue characteristics that are not currently identifiable using existing imaging modalities. After lung cancer, breast cancer remains the deadliest cancer for women, taking the lives of approximately 40,200 women in 2001 according to National Cancer Institute. There were 192,000 new breast cancer cases in 2001. Approximately 28 million women in the US are screened for breast cancer each year.

A high percentage of breast cancers are not detected at the screening stage. Studies show that 20% to 50% of breast cancers go undetected at the screening stage. The motivation for early detection is great: breast cancer detected in the early stage has an average cost of treatment of $11,000 and a 5 year survival rate of approximately 96%, while late stage breast cancer costs $140,000 on average to treat and the 5 year survival falls to 20%. Medical professionals often rely on expensive biopsies to determine cancerous tissues. These procedures are neither fast nor patient-friendly. Radiation treatment of cancerous tumors is applied broadly and excessively throughout the region of the tumor to insure complete cancerous cell destruction. Clearly, there is a need for better imaging technologies for breast cancer detection and for real-time tracking of cancer cell destruction during radiation treatment procedures.

X-ray mammography is the preferred modality for breast cancer detection. With the development of digital systems, and the use of computer-aided diagnosis (CAD) that assists physicians in identifying suspicious lesions by scanning x-ray films, a large increase in mammography system sales is expected. However, as noted previously, a large number of cancers are not detected using x-ray mammography, and to reduce x-ray exposure, breast compression techniques are used which make the examination painful.

After a suspicious lesion is found, the standard procedure is to perform a biopsy. Surgical biopsy is recommended for suspicious lesions with a high chance of malignancy but fine-needle aspiration cytology (FNAC) and core biopsy can be inexpensive and effective alternatives. Both FNAC and core biopsy have helped to reduce the number of surgical biopsies, sparring patients anxiety and reducing the cost of the procedure. However, core biopsies have often failed to show invasive carcinoma and both FNAC and core biopsies can result in the displacement of malignant cells away from the target—resulting in misdiagnosis.

According to the American Cancer Society, approximately 80% of breast biopsies are benign. Because of this, new less invasive technologies have been developed including: terahertz pulse imaging (TPI); thermal and optical imaging techniques including infrared; fluorescent and electrical impedance imaging. For the most part, these technologies are being pursued as an adjunct to traditional imaging modalities including computed tomography, magnetic resonance imaging, positron emission tomography, ultrasound and hybrid systems such as PET-CT.

The biochemical properties of cancerous cells versus normal cells are characterized by three factors: increased intracellular content of sodium, potassium, and other ions; increased intracellular content of water; and a marked difference in the electrochemical properties of the cell membranes. The increased intracellular concentrations of sodium, potassium and other ions results in higher intracellular electrical conductivity. Likewise, the increased water content results in higher conductivity when fatty cells surround the cancerous cells, since water is a better conductor than fat. And finally, the biochemical differences in the cell membranes of cancerous cells result in greater electrical permittivity.

A study of breast carcinoma described three separate classifications of tissue: tumor bulk, infiltrating margins, and distant (normal) tissue. The center of the lesion is called the tumor bulk and it is characterized by a high percentage of collagen, elastic fibers, and many tumor cells. Few tumor cells and a large proportion of normally distributed collagen and fat in unaffected breast tissue characterize the infiltrating margins. Finally, the distant tissues (2 cm or more from the lesion) are characterized as normal tissue.

The characterization of cancerous tissue is divided into two groups: in situ and infiltrating lesions. In situ lesions are tumors that remain confined in epithelial tissue from which they originated. The tumor does not cross the basal membrane, thus the tumor and the healthy tissue are of the same nature (epithelial). The electrical impedance of an in situ lesion is thus dependent on the abundance of the malignant cells that will impact the macroscopic conductivity (which is influenced by the increase in sodium and water) and permittivity (which is influenced by the difference in cell membrane electrochemistry).

By contrast, infiltrating lesions are tumors that pass through the basal membrane. The malignant tissue has a different nature than normal tissue (epithelial vs. adipose). Epithelial tissue is compact and dense. Adipose tissue is composed of large cells that are mostly triglycerides. These structural differences have the following impact. First the normal tissue has a lower cellular density. Second, cell liquid of normal tissue is not as abundant as epithelial cells. Generally the radiuses of epithelial cells are less than adipose cells, from which we conclude that the radius of cancerous cells is less than for normal cells. The impact on the fractional volume of cancerous cells vs. normal cells is that the fractional volume of cancerous cells is greater than for normal cells. The reason is that the epithelial population is higher than for normal, adipose cells. Finally, we note that intracellular conductivity of cancerous cells is greater than for intracellular conductivity of normal cells. Also, extracellular conductivity is higher because of the abundance of the extracellular fluid (because of larger gaps between normal and cancerous cells). Thus, the conductivity of the infiltrated tissue will be greater than for normal tissue.

Since the 1950's several researchers have measured and tabulated the electrical properties of biological tissues. The electrical properties (conductivity and permittivity) of human tissues exhibit frequency dependence (dispersion). There are three dispersion regions (α, β, and γ) at frequencies ranging from D.C. to 1 GHz. These dispersions in tissues are dependent on the number of cells, the shape of the cells, and their orientation, as well as the chemical composition of the tissue (i.e. composition and ionic concentrations of interstitial space and cytoplasm).

Various studies show that the values of biological tissues resistivities vary for a host of reasons. Cancerous tumors, for instance, possess two orders of magnitude (factor of 100) higher conductivity and permittivity values than surrounding healthy tissue. The application of medical treatments also produces a change in the electrical properties of tissue. For muscle tissue treated with radiation measurable changes to tissue impedance is reported. Significant changes occur in electrical impedance of skeletal muscle at low frequencies during hyperthermia treatment, and this change of electrical properties foreshadows the onset of cell necrosis.

Electrical impedance tomography (EIT) is a process that maps the impedance distribution within an object. This map is typically created from the application of current and the measurement of potential differences along the boundary of that object. There are three categories of EIT systems: current injection devices, applied potential devices, and induction devices. Henderson and Webster first introduced a device known as the impedance camera that produced a general map of impedance distribution. The Sheffield System and its incarnations were the first generation EIT system. In the later 80's, Li and Kruger report on an induced current device. In such a system, a combination of coils is placed around the object under test. A changing current in the coils produces a varying magnetic field that in turn induces a current in the object under test. As with the other drive method, electrodes are placed on the boundary of the object to measure the potential drops along the boundary.

Such electrical property imaging techniques are often referred to as “impedance tomography.” Most conventional electrical property imaging techniques are based on the premises that: 1) electrodes, or sensors, should be attached directly to the sample to be measured (for medical applications, the sample is a human body), and 2) current is injected sequentially through each electrode into the sample and the subsequent voltages measured. Therefore, these conventional EIT imaging techniques implement a “constant current/measured voltage” scheme.

In a departure from such conventional electrical property imaging techniques, U.S. Pat. No. 4,493,039 disclosed a method in which sensors are arranged in an array outside the object to be measured and during imaging of a sample, ac voltages are applied at a fixed amplitude while the current is measured. This approach, which is sometimes referred to as electrical property enhanced tomography (EPET), was further improved as described in pending patent application WO 99/12470 by filling the space between the object and the sensor array with an impedance matching medium. In addition, a technique for computing the internal charge distribution based on the measured surface charges is described and referred to as the charge-charge correlation technique. The charge-charge correlation technique requires position information of the internal structures derived from an associated imaging system such as an MRI or CT system. The charge-charge correlation technique also requires an approximation of the local gradient of the potential field. Despite these requirements, the present invention improves upon the prior methods both in the accuracy of the results calculated and in the time required for computation. The present invention not only produces consistently accurate values of the electrical characteristics of an object, but also requires substantially less time to compute these values.

### SUMMARY OF THE INVENTION

The present invention solves the problems associated with prior electrical parameter imaging techniques by providing a new electrical property imaging method which substantially reduces the processing time required for image reconstruction. More specifically, a structural image of the object is acquired to locate boundaries therein between different tissues, and then a series of measurements are made wherein voltages are applied to the surface of the object and resulting surface currents are measured. An image is reconstructed using this information which includes: predicted currents at an internal boundary; and calculating a first contrast ratio at the internal boundary which indicates the ratio between electrical characteristics sharing that common boundary. From the contrast ratio and the known electrical characteristics of tissues to one side of the boundary, the electrical characteristics of the tissues on the other side of the boundary are calculated. These calculations are made at successive boundaries using previously calculated electrical characteristics.

An object of the invention is to reduce the electrical property image reconstruction time with the combination of measured surface currents and positional information obtained from a secondary imaging modality. The positional information may be, for example, the locations of the boundaries of internal structures as determined by an MRI or CT system. Using the knowledge of the location of the boundaries of these internal structures, the electrical characteristics of each of these regions can be determined.

A more specific object of the invention is to provide an electrical property image reconstruction method which is flexible and can be used in many different EPET configurations. For example, it can be employed in an EPET system in which only the outer contour of the subject is measured or known, or it can be used in an EPET system that employs sophisticated computed tomography equipment to provide detailed information about the outer contour and internal structures of the subject.

The foregoing and other objects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram showing an exemplary computer system useful for implementing the present invention;

FIG. 2 is a planar view of a closed volume in space;

FIG. 3 is a planar view of a closed volume in space showing the relationship between the measured exterior total charges Qⱼ and the interior total charges qₖ;

FIG. 4 is a planar view of a closed volume in space being measured by a conventional electrical property imaging technique;

FIG. 5 is a block diagram of the preferred embodiment of an electrical properties imaging system which employs the present invention;

FIG. 6 is a circuit diagram of a voltage driven circuit which forms part of the system of FIG. 5;

FIG. 7 is a schematic diagram of one embodiment of a measurement array support which forms part of the system of FIG. 5;

FIG. 8 is a flow chart of a data acquisition program performed by the computer controller in FIG. 5; and

FIG. 9 is a flow chart of an image reconstruction program performed by the computer controller of FIG. 5.

FIG. 10 is a planar view of a closed volume in space containing distinct regions of different electrical properties.

### DETAILED DESCRIPTION OF THE PREFERRED EMBODIMENT

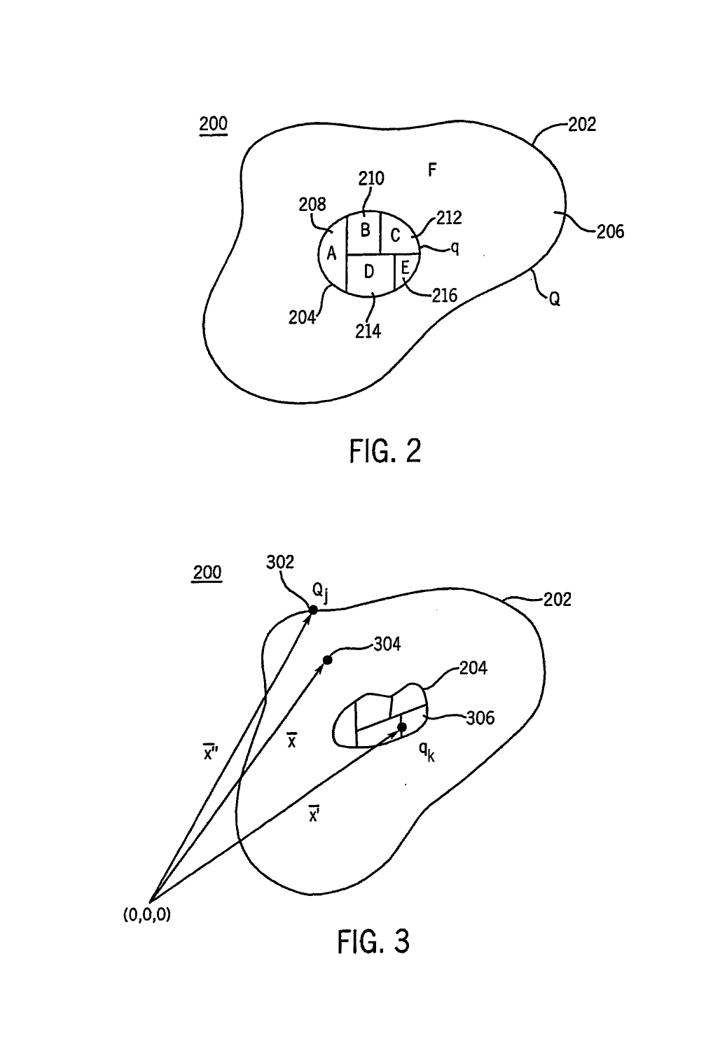

The underlying mathematical theory of the imaging technique of the present invention will now be described with reference to FIGS. 2-4. FIG. 2 is a planar view of a closed volume space 200 surrounded by a surface 202 that contains a sample 204 and an interior region F 206, such that region F 206 is the space between the sample 204 and the surface 202. The sample 204 comprises a plurality of connected subregions which for convenience are labeled: subregion A 208, subregion B 210, subregion C 212, subregion D 214, and subregion E 216. Each subregion 208-216 may be composed of a different material, such as different tissues in a human subject.

When an electromagnetic field at some specified frequency (ω) is applied to the sample 204 in the closed volume space 200, a total charge is produced only where the electrical properties change, such as at the boundaries between each subregion 208-216 of the sample 204 where there is a dissimilarity in the dielectric constant and conductivity electrical properties of each subregion 208-216. These total charges will in turn induce a redistribution of the total charges on the surface of the closed volume space 200. It is assumed that these induced charge distributions result from both free charges (free to move individually) as well as polarization charges located on the surface 202 of the closed volume space 200. The charges on the surface 202 are also total (free plus polarization) charges wherein the total charge on a point on the surface 202 is indicated with a capital “Q”, while the total charge on a point in the interior of the closed volume space 200 is indicated with a small “q.” It is important to note that the measurement of the total charge Q can involve either an actual measurement of the charge Q or the charge Q as derived from a small increment of the electrical current, I, which is the rate of change of the charge Q with time.

The total charge Q at a point on the surface 202, and the total charge q at a point in the interior can be connected via electromagnetic theory. When time varying electric fields are applied to electrical media they induce currents in the media. These currents in turn produce time varying magnetic fields that can add induced electric fields to the applied electric field via Faraday's law. This extra contribution to the electric field is negligible at low frequencies and can be ignored. We will use this so-called quasi-static approximation. The fundamental theorem of electrostatics shows that an interior total charge q and a total charge Q on the surface 202 are uniquely related.

FIG. 3 is a planar view of the closed volume space 200 showing the relationship between the total charge Q at a point 302 on the surface 202 and a total charge q at a point in the interior that are connected via The Greens Function. Specifically, The Greens Function connects a total charge Q on the surface 202 at point j with an interior total charge q at point k:
qₖ⇄Qⱼ

This relationship provides the desired information about the electrical properties of the interior subregions 208-216 of sample 204. FIG. 3 illustrates the coordinate system and some of the relevant geometry used in this discussion. The notation used in the coordinate system for the field point 304, the source point 306 and surface point 302 are X̅, X̅ prime ( X̅′), and X̅ double prime ( X̅″) respectively. By associating the total charges q inside the sample 204 at the source point 306 with the total charges Q at the surface point 302, an enhanced image of the interior of the sample 204 can be generated. Therefore, the position at which the electric field is measured is field point 304.

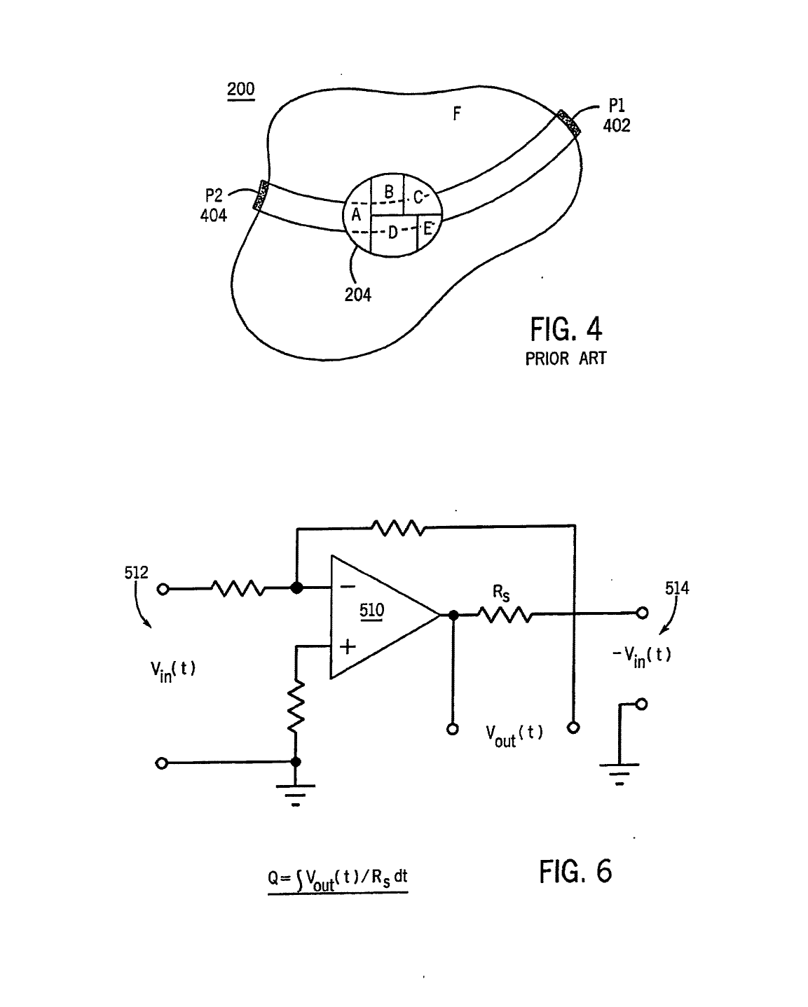

The imaging technique of the present method differs significantly from the conventional electrical property imaging techniques. FIG. 4 is a planar view of a closed volume space 200 being measured by such conventional imaging techniques. The electrical properties of the sample are represented by a network of lumped circuit elements. With such a method, currents are injected at known places, e.g., P1 402, on the surface 202 of the closed volume space 200 and extracted at known places, e.g., P2 404. The voltages on the surrounding sensors are then measured and the lumped circuit impedances are computed from the set of current-voltage measurements. In contrast, the technique of the present invention allows one to fully describe the wave-like nature of the electric fields in the closed volume space 200 and the measuring volume and does not require any specific assumption regarding the structure of a lumped circuit element network or of the equivalent circuits used to characterize the subregions 208-216 of the sample 204 being measured.

Applying the Maxwell Equations of electromagnetic theory to the problem as just described results in Equation 1A:
∇·((σ+iωε₀εᵣ)∇Φ)=0 (1A)
where: σ=conductivity
i=imaginary number
ω=frequency of potential field
εᵣ=relative dielectric constant
ε₀=dielectric constant of free space
Φ=potential.

In addition, a standard result of electromagnetic theory is the connection between the potential, (Φ), and the total charge density, ρ, known as the Poisson Equation, Equation 1B:

$${∇}^{2} Φ = - \frac{{ρ}_{total}}{{ɛ}_{0}} \qquad ( 1 B )$$

where ρ_{Total} is the volume total charge density. The field E is obtained from the following equation:
E=−∇Φ (1C)

The Equations 1A and 1B show that the scalar potential phi (Φ), the charge densities that are important are related to the total charge, i.e., the free charge plus polarization charge.

Other methods for imaging the electrical properties attempt to compute the dielectric constant and conductivity of each region directly from the measurements. We compute the current at the boundary between regions of different electrical characteristics as an intermediate step. One advantage of seeking the currents, which are determined by the internal charges, rather than going directly for the conductivity or dielectric constant is that one can see that the currents, which totally govern the electrical picture, appear essentially only at boundaries that exist at discontinuities within the object, thus there are far fewer values to compute. Equation 2 below shows this since the gradient of the conductivity and the gradient of the dielectric constant contribute to the total charge density. Therefore, total charge depends on the rate with which the conductivity and the dielectric constant change with distance.

$${ρ}_{total} = \frac{∇ σ + ⅈ ω ∇ ( {ɛ}_{0} {ɛ}_{r} )}{σ + ⅈ ω {ɛ}_{0} {ɛ}_{r}} \cdot {ɛ}_{0} \cdot ∇ Φ \qquad ( 2 )$$

A standard theorem in electromagnetic theory is the Uniqueness Theorem. The Uniqueness Theorem for the quasistatic case states that if the potential or its normal derivative is known on a surface surrounding a closed volume, then the potential at a field point 304 can be uniquely determined. It is important to note that both the potential and the normal derivative of the potential need not be known. In fact, the problem would be over determined if both were known. While it is possible to define the problem with the potential known on some portion of the bounding surface and the normal derivative on other portions, Equation (3) below considers the simple case where the potential on the surface 202 is known. This is known as the Dirichlet boundary condition.

Equation 3 is the solution to Poisson's Equation (Equation 2) using the Green's Function.

$$Φ ( \overline{X} ) = \frac{1}{4 {πɛ}_{0}} [ {\int }_{V} ρ ( {x}^{′} ) {G}_{D} ( \overline{X} , {\overline{X}}^{′} ) {ⅆ}^{3} {X}^{′} - {ɛ}_{0} {∮}_{S} {Φ}_{S} ( {\overline{X}}^{′} ) \frac{\partial {G}_{D}}{\partial {n}^{′}} ⅆ S ] \qquad ( 3 )$$

Where G_{D} is the Dirichlet Green's Function, dτ is an element of volume, dS is an element of surface surrounding the volume τ, and the notation

$$\frac{\partial }{\partial {n}^{′}}$$

indicates the derivative normal to the surface, S.

Equation 3 is the potential at the field point 304 as determined by the total charge q on the interior and the potential on the surface 202, exactly as the Uniqueness Theorem predicts. The solution is obtained in the terms of a geometrical function, the Green's Function, which is a standard treatment. When a sample 204 is present, both the volume integral over the total charge q density and the surface integral over the surface 204 are present. If the same potential distribution on the surface is considered but with no sample present, then the charge density goes to zero but the surface integral remains the same. The surface term (the second integral in Equation 3) is unchanged by inserting the sample 204 because the voltage is set to pre-determined values on the surface 202 and kept at those values before and after inserting the sample 204. Because of this, when the two terms are subtracted, the remaining expression involves only the Green's Function (which is a known quantity for a given shape of the array of measuring sensors) and the charge density. Therefore, it is convenient to use the difference in the potential between the case when a sample 204 is inserted and when a sample 204 is not inserted between the sensors. This potential difference can be related to the charges at the surface 202 by taking the normal derivative of the potential difference to produce the normal component of the electric field since, by Gauss's law, the normal component of the field near a conducting surface is directly proportional to the charge per area on that surface. We then change from a continuum model to a sum over discrete charges and Equation (4) below then shows that those charges Qⱼ at the surface 202 labeled by the index “j” will be related to the charges qₖ on the interior labeled by the index “k” by a matrix element involving both “j” and “k” wherein the connecting matrix element is simply the normal derivative of the Green's Function:

$$δ {Q}_{j} ( \overline{X} ) = - \frac{ⅈ ( σ + {ⅈωɛ}_{0} {ɛ}_{r} )}{4 {πωɛ}_{0}} \cdot δ S \cdot {\sum }_{k = 1}^{N} \{ ∇ {G}_{D} ( \overline{X} , {\overline{X}}_{k}^{′} ) \cdot {\hat{n}}_{k} \} δ {q}_{k} \qquad ( 4 )$$

Where δqₖ is an elemental charge at the internal location (xₖ′, yₖ′), \hat{n}ₖ is a unit vector normal to the surface of the surface 202, and δS is an elemental surface area on an electrode in the above equation. Since the charge builds up on the surface only where the applied electric field is normal to the surface, at least two orientations of the electric field are preferred to obtain information about how charges build up at all points on the surface 202.

As will be described in more detail below, the subject to be imaged is placed in a measurement array which enables a sinusoidal voltage of a desired frequency and 15 or less volts rms to be applied to the surface of the subject to establish an electric field E̅ through the subject. The surface charges Qⱼ that result from this applied field are measured. The surface charge measurement may be repeated with the applied electric field oriented in different directions and it may be repeated at different frequencies from 10 KHz to 10 MHz.

Equation 5 shows the Green's Function expanded as a complete set of orthogonal functions, the result of which is a sum over the parameter “L” which appears inside the sine function in Equation 5. Multiplying the appropriate sine function for given value “L”, and summing up over one side of the measurement array, the sum over “L” is eliminated, thereby leaving just one term. This result occurs because of the orthogonality property of sine and cosine functions. The accuracy can be further improved by adding the results from corresponding measurements on opposite sides of the measurement array resulting in the equation for a given value of “L” for the Fourier Transform (the sine transform) as shown in Equation 6. The variable A_{L} in equation 6 is the sine transform coefficient in the sine series approximation of the boundary charge distribution, Q, and is shown in equation (7).

$$\begin{matrix}{G}_{D} ( \overline{X} , {\overline{X}}^{′} ) = \frac{8}{π a c} {\sum }_{L = 1}^{{L}_{max}} \frac{sin ( \frac{L π \overline{X}}{a} ) sin ( \frac{L π {\overline{X}}^{′}}{a} ) sinh ( \frac{L π}{a} {\overline{Y}}^{′} ) sinh ( \frac{L π}{a} ( b - {\overline{Y}}^{′} ) )}{\frac{L π}{a} sinh ( \frac{L π}{a} b )} & ( 5 ) \\ ST \{ δ Q ( \overline{X} ) , L \} = {A}_{L} = \frac{2}{a} {\int }_{0}^{a} δ Q ( \overline{X} ) sin ( \frac{L π \overline{X}}{a} ) ⅆ x & ( 6 ) \\ {A}_{L} = - 2 \frac{ⅈ ( σ + {ⅈωɛ}_{0} {ɛ}_{r} )}{ω {ɛ}_{0}} ( \frac{δ S}{a c} ) {\sum }_{k = 1}^{N} \{ sin ( \frac{L π {\overline{X}}_{k}^{′}}{a} ) \frac{sinh ( \frac{L π}{a} ( b - {\overline{Y}}_{k}^{′} ) )}{sinh ( \frac{L π}{a} b )} \} δ {q}_{k} & ( 7 )\end{matrix}$$

Equation 7 above can be converted into a matrix expression and one embodiment of a weighting function, B(L), can be defined, as is shown below in Equations 8 and 9 respectively:

$$\begin{matrix}( \begin{matrix}{A}_{1} \\ {A}_{2} \\ ⋮ \\ {A}_{{L}_{max}}\end{matrix} ) = - 2 \frac{ⅈ ( σ + {ⅈωɛ}_{0} {ɛ}_{r} )}{ω {ɛ}_{0}} ( \frac{δ S}{a c} ) ( \begin{matrix}{B}_{1} ( 1 ) & {B}_{2} ( 1 ) & … & {B}_{N} ( 1 ) \\ {B}_{1} ( 2 ) & ⋰ & & \\ ⋮ & & & \\ {B}_{1} ( {L}_{max} ) & & & {B}_{N} ( {L}_{max} )\end{matrix} ) ( \begin{matrix}δ {q}_{1} \\ δ {q}_{2} \\ ⋮ \\ δ {q}_{N}\end{matrix} ) & ( 8 ) \\ {B}_{k} ( L ) = sin ( \frac{L π {\overline{X}}_{k}^{′}}{a} ) \frac{sinh ( \frac{L π}{a} ( b - {\overline{Y}}_{k}^{′} ) )}{sinh ( \frac{L π}{a} b )} & ( 9 )\end{matrix}$$

Where a, b, and c are, respectively, the length in the x-direction, width in the y-direction, and depth in the z-direction of the measurement support array in the preferred embodiment of the present invention.

The internal charges, qₖ, can be related to the desired electrical characteristics of the object to be imaged by defining a contrast ratio, κ, in Equation 11, which is a ratio of the electrical properties that meet at a common boundary. Since the charge distribution accumulates only on the bounding surface, the internal charge density is effectively a charge per unit area. Following this idea, Equation 2 can be evaluated at the boundary between regions of different electrical characteristics to yield equation 10:

$$\begin{matrix}δ q = ( \frac{κ - 1}{κ + 1} ) ( \frac{- {ɛ}_{0} δ S}{2} ) \hat{n} \cdot ∇ Φ & ( 10 ) \\ κ = \frac{{σ}_{2}^{*}}{{σ}_{1}^{*}} & ( 11 ) \\ {σ}^{*} = σ + {ⅈωɛ}_{0} {ɛ}_{r} & ( 11 A )\end{matrix}$$

Where σ₁* and σ₂* are the complex conductivities in a first and a second adjacent region, respectively, and ∇Φ is the gradient of the applied potential field which in the preferred embodiment is approximated using a finite difference method. From Equation 11A, it can be seen that for a material in which an alternating current is present, the complex conductivity of that material is dependent on the frequency of the applied potential field, ω. Due to this frequency dependence, the preferred embodiment of the invention collects data for applied fields of varying frequencies.

The procedure now is relatively simple. For each value of “L”, one equation can be produced each of which computes the predicted current pattern at the boundary between regions. By combining equations 7 and 10, an expression for the predicted current pattern for an applied potential with frequency, ω, can be produced. The expression for the predicted current is shown in Equation 12 below with the new sine transform coefficients given in Equation 13:

$$\begin{matrix}{I}^{p} ( \overline{X} ) = {\sum }_{L = 1}^{{L}_{max}} {A}_{L}^{′} \cdot sin ( \frac{L π \overline{X}}{a} ) & ( 12 ) \\ {A}_{L}^{′} = - ( {σ}_{1}^{*} \frac{δ x}{a} ) {\sum }_{k = 1}^{N} {B}_{k} ( L ) \cdot ( δ {area}_{k} \cdot {\hat{n}}_{k} \cdot ∇ Φ {\vert}_{( {x}_{k} , {y}_{k} )} ) & ( 13 )\end{matrix}$$

Using the process described below, an accurate representation of the electrical characteristics in the interior of the object can be determined. First, measurements of the current through a plurality of sensor electrodes are acquired in a measurement chamber filled with an impedance matching solution of known complex conductivity. This data is stored and the object to be imaged is then placed in the measurement chamber and another set of current patterns is acquired. The difference of these two measured currents is then calculated using Equation 14. The predicted current pattern on a given boundary between regions is computed and with the measured current difference, I^{m}, described above, the contrast ratio can be determined by solving Equation 15. Once the contrast ratio is known, Equation 11 can be applied to sequentially obtain the complex conductivities for every region in the interior of an object. This process remains applicable in the case where a plurality of boundaries are encountered.

$$\begin{matrix}{I}^{m} = {I}_{empty}^{m} - {I}_{subject}^{m} & ( 14 ) \\ {I}^{m} = ξ \cdot {I}^{p} & ( 15 ) \\ ξ = \frac{κ - 1}{κ + 1} & ( 16 )\end{matrix}$$

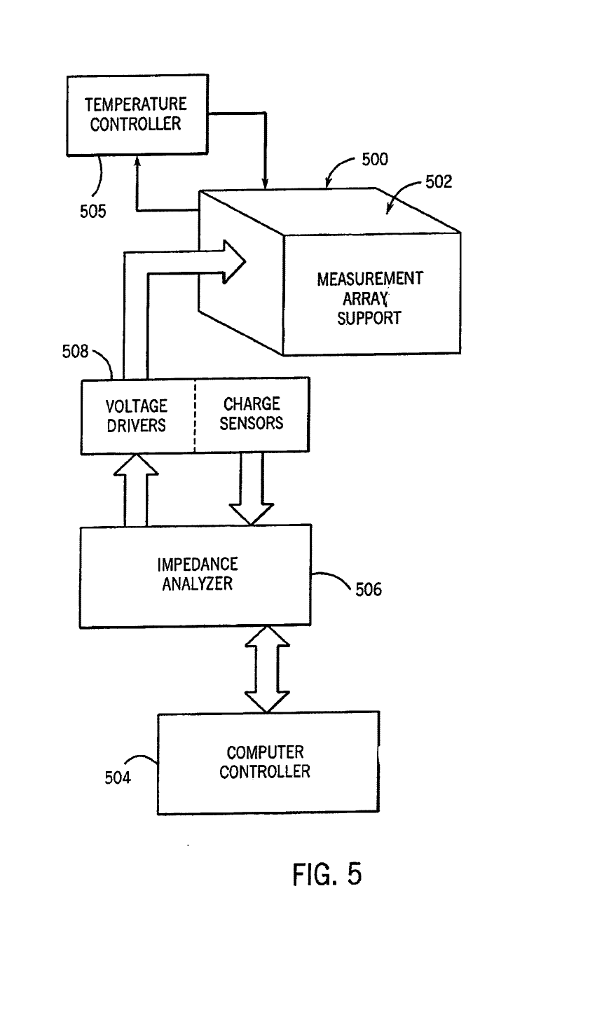

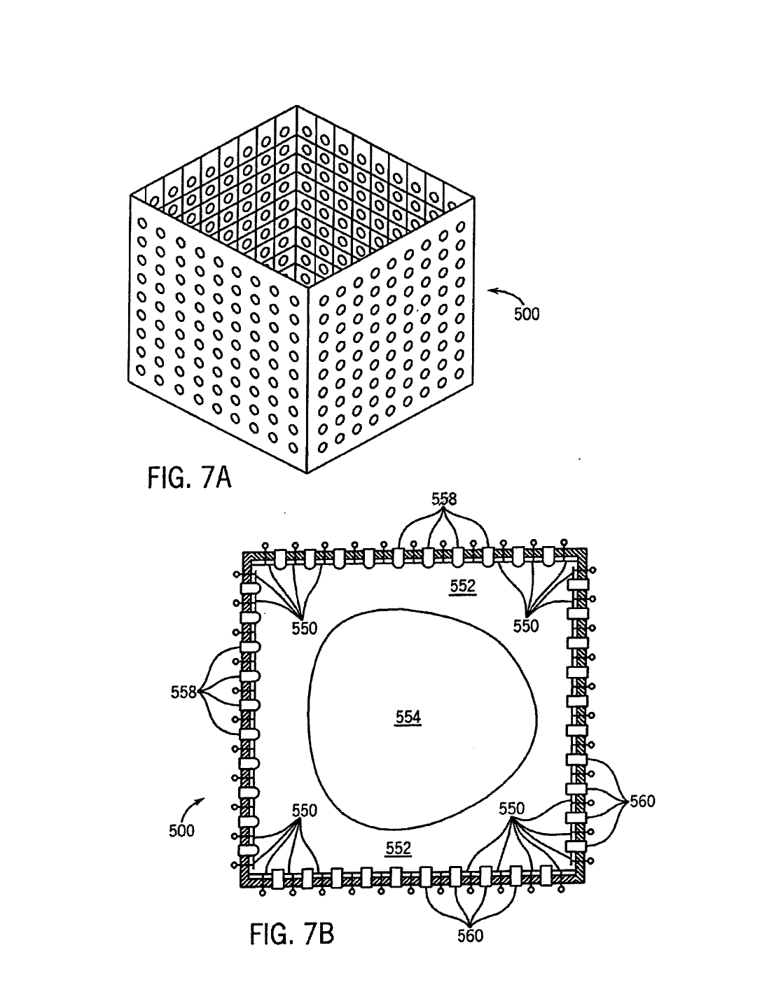

A preferred system for acquiring the surface charge data and producing therefrom an image indicative of the electrical characteristics of the subject is shown in FIG. 5. It includes a measurement array support structure 500 that is illustrated in more detail in FIGS. 7A and 7B and described in detail below. The support structure 500 has four vertical sides and a bottom which forms a container that is filled with a saline water solution of known electrical properties that are matched as closely as possible to the electrical properties of the subject. The subject to be imaged is inserted through the open top 502. When used to image the breast, the support structure 500 is mounted beneath an opening in a patient table and the breast is aligned to hang down into the container.

A sophisticated imaging system may be employed to acquire detailed geometric information about the outer contour and internal structures of the object to be imaged. For example, an x-ray tomosynthesis system such as that disclosed in U.S. Pat. No. 6,611,575 can acquire a three-dimensional image of the subject. Preferably, such image data is acquired while the patient is positioned for the EPET examination and it is automatically registered with the position of the measurement array support 500. If not, a separate image registration step is required to position the geometric structures revealed in the a priori 3D image in the same position within the support 500 as the subject being examined.

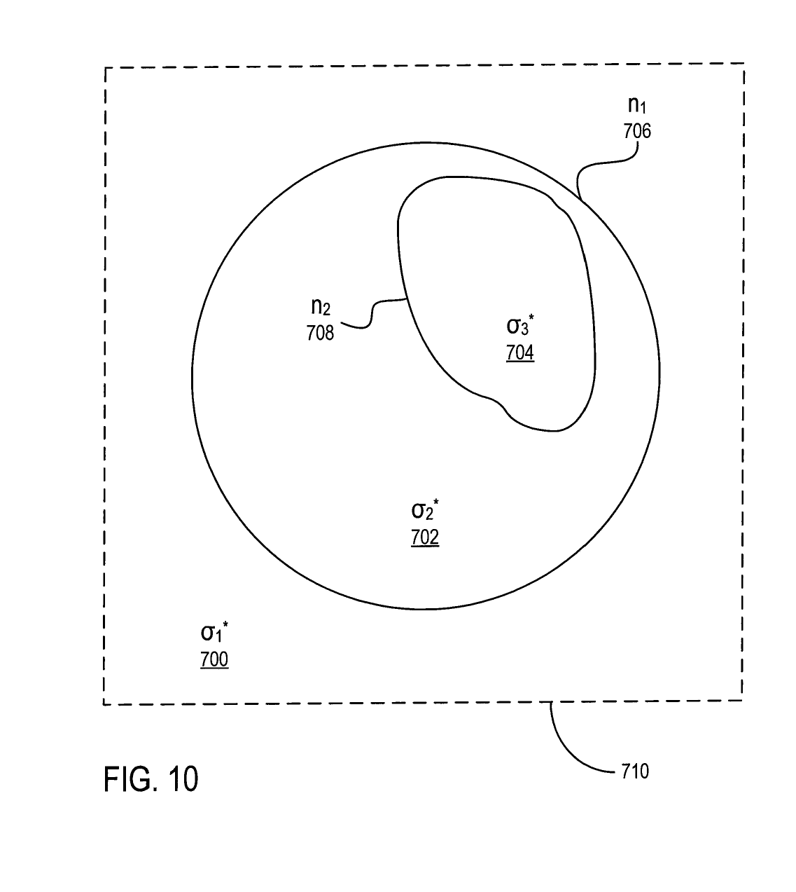

Referring particularly to FIG. 10 as an example of an object to be examined, a closed volume of space 700 with complex conductivity σ₁* is bound by a non-distinct boundary 710 and contains a closed volume of space 702 with complex conductivity σ₂* bound by boundary n₁706 and further containing another closed volume of space 704 with complex conductivity σ₃* which is bound by boundary n₂708. In the preferred embodiment of the present invention, the closed volume 700 is an impedance matched solution with a known complex conductivity σ₁*, and is bound by a measurement array support 500. The positions of the internal boundaries n₁706 and n₂708 are determined by the secondary imaging system such as an MRI or CT system. This structural anatomic image which indicates the position of internal boundaries between different tissues is obtained contemporaneously with the electrical property data. The present invention determines the electrical characteristics σ₂* and σ₃* in this exemplary object.

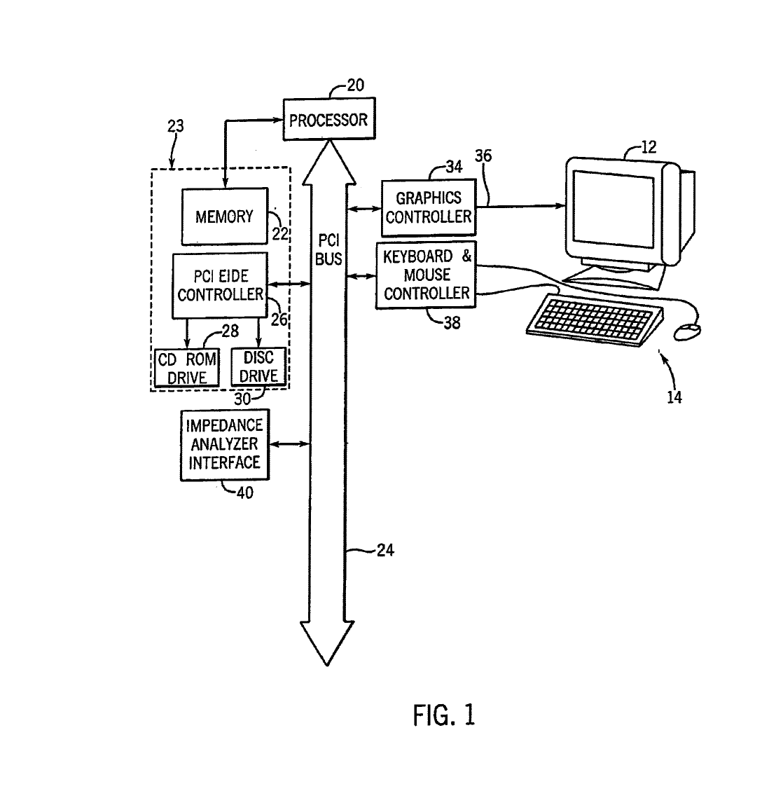

The system is controlled by a computer controller 504 which is shown in more detail in FIG. 1 and described below. It operates an impedance analyzer 506 to apply voltages to the separate elements of a charge measurement array through voltage drivers 508, and it measures the resulting charge Q at each of these elements. One embodiment of an impedance analyzer 506 is commercially available from Solartron Analytical under the trade name “1260 Impedance/Gain Phase Analyzer”. It is operated using its “Z plot” software that is run on the computer controller 504.

The voltage drivers and charge sensors are shown in detail in FIG. 6. The operational amplifier 510 is operated as an inverter with unity gain between its input terminals 512 and a pair of outputs 514 that connect to a charge measurement array element. The voltage drop across a series connected output resister Rₛ serves as the output to the analyzer 506 and is used to calculate the resulting surface charge Qⱼ at the charge measurement array element to which the outputs 514 connect.

To maintain the accuracy of the measurements the temperature of the saline solution in the measurement array support structure 500 is controlled. This is accomplished by a temperature controller 505 which operates a heating element (not shown) in the support 500 in response to a signal received from a temperature sensor (not shown) which is also in the support 500. Preferably, the temperature is maintained at body temperature for the comfort of the patient.

Referring particularly to FIGS. 7A and 7B, one preferred embodiment of the measurement arrays support structure 500 includes 2D arrays of metal elements 550 disposed on all four sides of the container. These elements 550 are square metal electrodes that connect to the outputs 514 of corresponding voltage drivers 508. They are in electrical contact with the saline solution medium 552 that surrounds the subject 554. The voltages applied to these elements 550 establish an electric field E within the container and throughout the subject 554, and they accumulate a surface charge Qⱼ that is dependent on the electrical characteristics of the subject 554. In this preferred embodiment 225 elements 550 are disposed on each of the four sides and they are constructed of silver with a silver chloride coating.

Referring particularly to FIG. 1, a computer controller system includes a processor 20 which executes program instructions stored in a memory 22 that forms part of a storage system 23. The processor 20 is a commercially available device designed to operate with one of the Microsoft Corporation Windows operating systems. It includes internal memory and I/O control to facilitate system integration and integral memory management circuitry for handling all external memory 22. The processor 20 also includes a PCI bus driver which provides a direct interface with a 32-bit PCI bus 24.

The PCI bus 24 is an industry standard bus that transfers 32-bits of data between the processor 20 and a number of peripheral controller cards. These include a PCI EIDE controller 26 which provides a high-speed transfer of data to and from a CD ROM drive 28 and a disc drive 30. A graphics controller 34 couples the PCI bus 24 to a CRT monitor 12 through a standard VGA connection 36, and a keyboard and mouse controller 38 receives data that is manually input through a keyboard and mouse 14.

The PCI bus 24 also connects to an impedance analyzer interface card 40. The interface card 40 couples data to and from the impedance analyzer 506 during the data acquisition phase of the procedure. A program executed by the processor 20 controls the impedance analyzer 506 to apply voltages to the charge measurement array and to input data indicative of the resulting current.

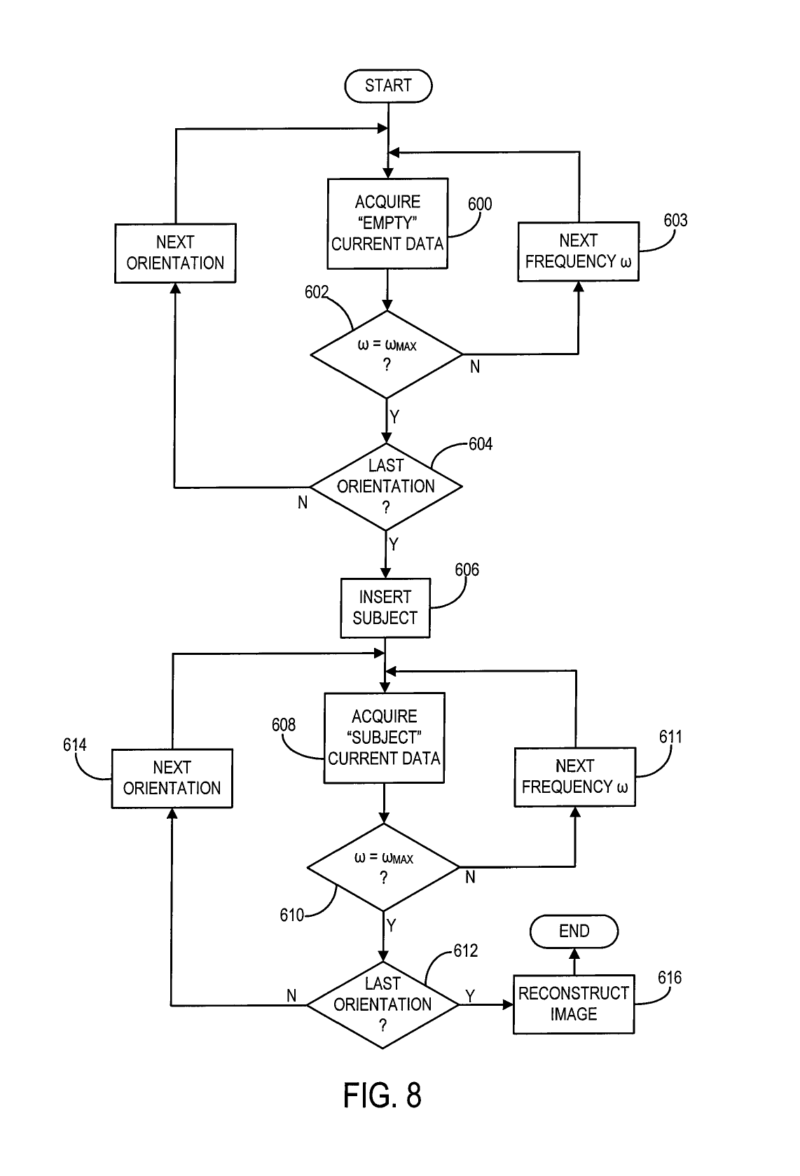

Referring particularly to FIG. 8, the procedure is comprised of an image acquisition phase and an image reconstruction phase. As indicated by process block 600, the first step in the image acquisition phase is to acquire current data I_{empty}^{m} without the subject in place. This “empty” current data is stored as a vector array which is needed during the reconstruction phase and it is acquired by applying voltages at a selected frequency to the measurement array 500 as described above. The resulting measured current I_{empty}^{m} that accumulates over a finite time interval are acquired. The system loops back at decision block 602 to collect current data by applying ωₘₐₓ different voltages each with different frequencies ω as indicated at process block 603. The system also loops back at decision block 604 to repeat the above measurements for each different E field orientation that is to be acquired.

The subject is then inserted into the measurement array support 500 as indicated at process block 606. A loop is then entered in which the surface current data I_{subject}^{m} is acquired at the prescribed frequencies and the prescribed E field orientations. The current data I_{subject}^{m} is acquired at process block 608 by applying voltages to the charge measurement elements 550 at the prescribed frequencies and reading the currents I_{subject}^{m} that accumulate at each element 550. The measurement is repeated at each prescribed frequency ω as indicated at process block 611 until the last frequency is employed as determined at decision block 610. As indicated at process block 614, the system then loops back to repeat these measurements at other E field orientations. Since charges build up on the surface of a boundary only where the applied E field is normal to the surface, the preferred embodiment of the invention employs at least two applied E field directions. In this manner, the build up of charges, and thus current, at each point on the internal boundaries of the object can be substantially well characterized. The voltage amplitudes applied to the charge measurement elements 550 are changed to reorient the direction of the electric field E that is produced in the measurement array support 500. When the surface charge data has been acquired for the last E field orientation as determined at decision block 612, the image reconstruction can begin as indicated at process block 616.

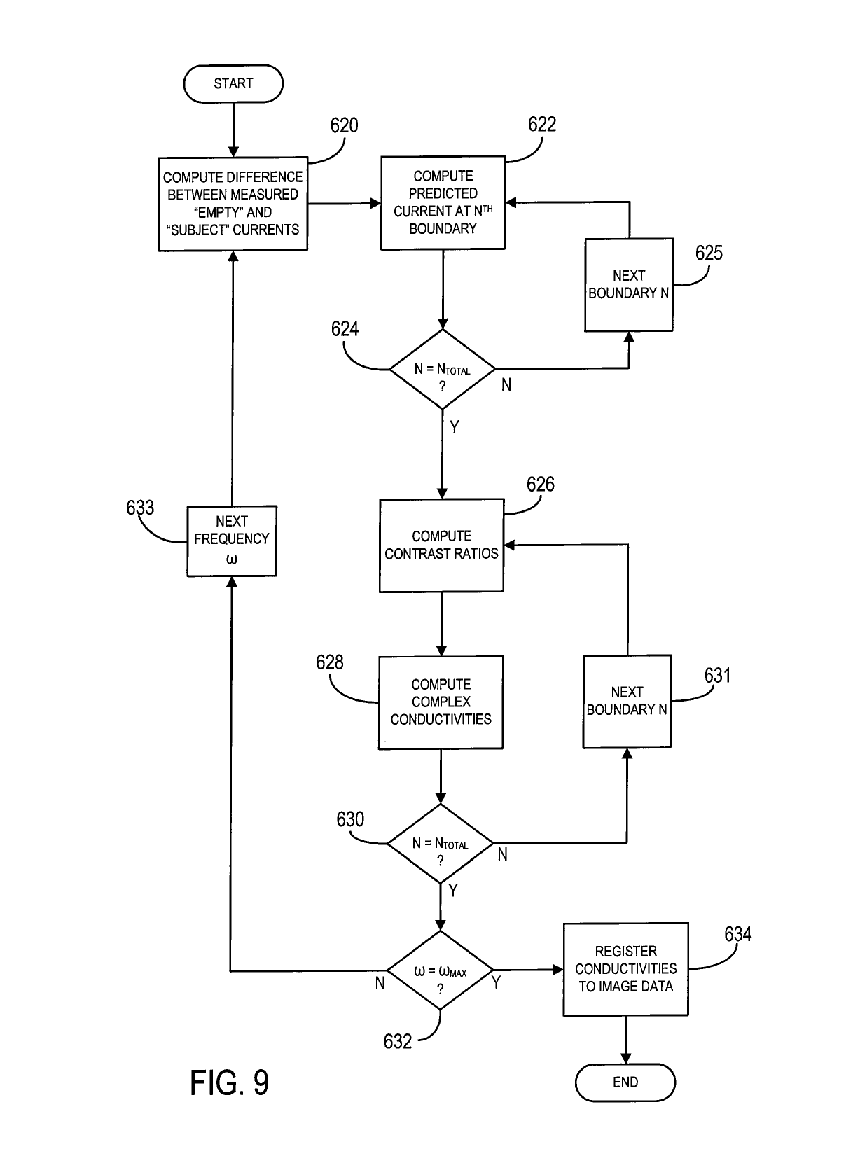

Referring particularly to FIG. 9, image reconstruction begins by using Equation 14 described above to compute the difference between the currents measured using the empty measurement array support 500 and the currents measured with the subject inserted as indicated in process block 620.

The next step as indicated at process block 622 is to compute the predicted current patterns for nₜₒₜₐₗ different boundaries, such as for the boundaries n₁706 and n₂708 in the example of FIG. 10 described above. Successive boundaries are chosen in process block 625, and the process is repeated until all boundaries have been processed as determined at decision block 624. In the preferred embodiment of the present invention, process block 622 is performed using equations 9, 12, and 13 described above. First, a weighting function Bₖ(L) is computed using the location information of the points (xₖ′,yₖ′) on the present boundary. Next, the predicted current at the boundary is computed using Equation 12 by first calculating each of L sine transformation coefficients, A_{L}′, wherein the location information of the points (xₖ′,yₖ′) on the boundary are used to determine the direction normal to the surface at each of those points on the boundary. The predicted boundary current data are stored in a matrix array having at least one matrix dimension equal to nₜₒₜₐₗ.

The next step as indicated by process block 626 is to compute the contrast ratios for each of the nₜₒₜₐₗ boundaries, which are then stored in a vector array. This computation is performed by first using the measured and predicted current data, I^{m} and I^{p} respectively, to solve Equation 15 described above. This computational step employs a well conditioned matrix inversion which can be performed using a regression method such as a least squares estimation, and thus exhibits a substantial reduction in processing time over prior methods. The parameter, ζ, solved for in Equation 15 can then be used to directly determine the contrast ratio through the simple algebraic relationship in Equation 16. A loop is then entered in which the complex conductivities of each different region are determined, as indicated by process block 628. This computation employs Equation 11 above at successive boundaries, and determines the complex conductivity of a region to one side of a boundary using the known or computed complex conductivity of the region to the other side of the boundary. The complex conductivity of successive regions are thus calculated as indicated at process block 631 until the complex conductivity of all regions are determined as indicated at decision block 630. In the example of FIG. 10, the complex conductivity σ₁* for the surrounding region 700 is known and is used to calculate the electrical characteristics σ₂* of region 702 using the calculated contrast ratio for boundary n₁706. Then, σ₂* is used to calculate the electrical characteristics σ₃* in region 704 using the calculated contrast ratio for boundary n₂708.

The entire process is then repeated for the next frequency ω as chosen in process block 633 until all of the prescribed ωₘₐₓ frequencies have been employed as determined in decision block 632. For each frequency, one set of measured currents I^{m} and one set of predicted currents I^{p} are used to compute one set of complex conductivities in the above manner. The set of these complex conductivities is then registered to the accompanying image obtained from the second imaging modality in process block 634 and the information about the complex conductivities obtained by applying voltages at a plurality of frequencies can be used to characterize different materials.

An advantage of the present invention over prior techniques is that an accurate and non-computationally intense set of the electrical characteristics of an object is produced by using an imaging modality such as MRI or CT and a quick and refined image reconstruction method. Furthermore, by employing applied potential fields at multiple frequencies, a range of electrical characteristics is produced, allowing for a detailed characterization of the nature of different tissues. This system is, therefore, a more desirable breast cancer screening device.

The present invention has been described in terms of one or more preferred embodiments, and it should be appreciated that many equivalents, alternatives, variations, and modifications, aside from those expressly stated, are possible and within the scope of the invention.

1. A method for obtaining information indicative of an electrical characteristic of an object, the steps comprising:
a) applying a voltage to the surface of the object with an array of sensor elements that make electrical connection with the surface;
b) measuring the current at each sensor element that results from the applied voltage;
c) obtaining an image of the object and its internal structures with a second imaging modality to locate boundaries therein between structures having different electrical characteristics;
d) computing a predicted current located at a boundary;
e) calculating from the measured current and the predicted current located at the boundary a contrast ratio; and
f) calculating from the first contrast ratio and known electrical characteristics of the region to one side of the located boundary, the electrical characteristics of the region to the other side of the located boundary.

2. The method as recited in claim 1 wherein steps d), e), and f) are repeated for other located boundaries in the object.

3. The method as recited in claim 1 wherein the contrast ratio in step e) is calculated by solving a linear relationship between the measured current and the predicted current using a linear regression method.

4. The method as recited in claim 1 wherein the predicted current is calculated in step d) by transforming the voltage applied to the surface of the object.

5. The method as recited in claim 4 wherein the transformation is a sine transformation and is performed by:
producing a weighting function for each point on the boundary;
calculating a sine transformation coefficient using each weighting function; and
summing the product of the sine transformation coefficients with a sine function.

6. The method as recited in claim 1 wherein steps a) and b) are repeated with different applied voltage frequencies and a set of electrical characteristics is calculated in steps d) through f) for each frequency.

7. The method as recited in claim 1 wherein step a) is repeated with the electrical field E produced by the applied voltage oriented in a different direction before measuring the current in step b).

8. The method as recited in claim 1 wherein the second imaging modality used in step c) is a computed tomography system.

9. The method as recited in claim 1 wherein the second imaging modality used in step c) is an x-ray imaging system.

10. An apparatus for acquiring electrical property data from an object which comprises:
a container which defines a cavity for receiving the object;
an array of metal electrodes supported by the container and disposed around the object;
means for applying voltages to the array of metal electrodes; and
means for measuring the resulting charge produced at each metal electrode.

11. The apparatus as recited in claim 10 in which a fluid having known electrical properties is contained in the container and makes contact with the surface of the object and the metal electrodes.

12. The apparatus as recited in claim 11 in which the fluid is a saline water solution that is matched to the electrical properties of the object.

13. The apparatus as recited in claim 10 which includes a temperature controller that maintains the temperature of the apparatus at a substantially constant temperature.

14. The apparatus as recited in claim 11 which includes a temperature controller that maintains the fluid at a substantially constant temperature.

15. The apparatus as recited in claim 10 in which the object is a breast of a human subject and the container is mounted to a supporting structure such that the breast extends downward into the cavity when the human subject is supported by the supporting structure.
